(19) United States
(12) Patent Application Publication
(10) Pub. No.: US 2021/0142876 A1
(43) Pub. Date: May 13, 2021

(54) Electronic Medical Cannabis Prescription Fulfillment Systems and Methods of Operating Thereof

(71) Applicant: 1961628 Ontario Corp., North York (CA)

(72) Inventors: PLA Mohan Rajika Cooray, Scarborough (CA); Kregg Nelson Coutinho, Scarborough (CA); Andrew Binh-Hao Lu, Mississauga (CA)

(21) Appl. No.: 16/906,609

(22) Filed: Jun. 19, 2020

Related U.S. Application Data
(60) Provisional application No. 62/864,724, filed on Jun. 21, 2019.

(51) Int. Cl. G16H 20/10; G16H 10/60; G16H 80/00; G06Q 10/08; G06Q 30/06

(52) U.S. Cl. CPC G16H 20/10; G16H 10/60; G06Q 30/0641; G06Q 10/087; G06Q 30/0609; G16H 80/00

(57) ABSTRACT

An electronic medical cannabis prescription fulfillment system, and method of operating thereof, are disclosed. The system includes an interface component to receive a medical cannabis prescription; and a processor operable to: identify a user account corresponding to a patient; generate an alert when no user account is identified, otherwise, automatically verify patient data with the stored user data; display at least some product data of each medical cannabis product available for purchase; receive a first purchase order directly from the patient to purchase a first medical cannabis product from a first licensed seller; complete the first purchaser order; receive a second purchase order to purchase a second medical cannabis product from a second licensed seller different from the first licensed seller; and in response to receiving the second purchase order, automatically validate the medical cannabis prescription and complete the second purchase order when the medical cannabis prescription is validated.

### CROSS-REFERENCE TO RELATED PATENT APPLICATIONS

This application claims the benefit of U.S. Provisional Application No. 62/864,724, filed on Jun. 21, 2019. The complete disclosure of U.S. Provisional Application No. 62/864,724, is incorporated herein by reference.

### FIELD

The described embodiments relate to an electronic medical cannabis prescription fulfillment system, and methods of operating thereof.

### BACKGROUND

Cannabis is an increasingly common form of medication and therapeutic product. Studies have shown that the use of cannabis may relieve symptoms and/or modify diseases associated with the following, including, but not limited to, nausea and vomiting resulting from chemotherapy, chronic pain, anxiety, post-traumatic stress disorder (PTSD), sleep disorders including insomnia, irritable bowel syndrome (IBS), dyspepsia, Crohn's disease, ulcerative colitis, spasticity and pain in multiple sclerosis (MS), rare forms of pediatric epilepsy such as Dravet Syndrome or Lennox-Gaustat Syndrome, seizures and epileptiform disorders, opioid withdrawal and dependence, alcohol withdrawal and dependence, drug withdrawal and dependence, depression (major depressive disorder), dysthymia, generalized anxiety disorder (GAD), generalized nausea (including morning and motion sickness), Human Immunodeficiency Virus (with or without AIDS) and some of its complications, weight loss, appetite simulation (anorexia nervosa and non-nervosa related disorders), functional medical disorders, variants of chronic pain including fibromyalgia, chronic fatigue syndrome, complex regional pain syndromes, migraines, neuropathic pain (diabetic and non-diabetic), quality of life in patients with pain/cancer/other disorders, spinal cord injuries and associated symptoms, and many more novel medical insights to come including the interaction of cannabinoids with healing (DNA repair, cellular regeneration and apoptosis defense mechanisms), senescence (anti-aging effects from anti-inflammatory properties from cannabinoids), cognitive enhancement through neurotrophic effect (i.e. Nootropic interaction with cannabinoids), anti-infective properties including anti-bacterial, anti-viral, anti-fungal, anti-parasitic, and anti-neoplastic effects that provide novel and/or neoadjuvant areas of therapeutics for human and non-human malignancies.

The medical effects of cannabis, like with many medications, vary from one individual (including humans and non-humans) to another due to a variety of reasons including but not limited to interactions of the following: ingesting organism genetics (human and non-human), ingested cannabinoid genetics (i.e. phytocannabinoid or non-phytocannabinoid including yeast derived), exogenous cannabinoid interaction with the endocannabinoid system including its interactions with CB-1, CB-2, and other cannabinoid (CB) receptors in both humans and non-humans, species inter-sex end-organ variations, weight (including body fat composition), method and route of delivery (e.g., through inhaling, oral, etc.), dose per mg/kg delivered, bioavailability, digestion/absorption/circulation pharmacokinetics and pharmacodynamics (including fat soluble gastrointestinal absorption), cytochrome p450 liver metabolism, cytochrome p450 drug-drug interactions, unknown user-sensitive and user-specific end-organ effects associated with an organism-specific therapeutic window of cannabinoids, exogenous and endogenous cannabinoid interaction (i.e. neurotransmitter reuptake inhibition), volume of distribution (fat-soluble storage), fat-soluble exogenous cannabinoid clearance and excretion. The two main naturally occurring cannabinoids identified in the human endocannabinoid system that primarily act as neurotransmitters in the synaptic clefts of the cerebral cortex and possibly elsewhere (including but not limited the central nervous system, peripheral nervous system (including the enteric nervous system), and the autonomic nervous system (sympathetic and parasympathetic nervous systems) are Anandamide and 2-archidonoylglycerol (2-AG).

The cannabis plant (spp. Cannabis Sativa, Cannabis Indica, Cannabis Ruderalis) produce over 140 naturally occurring phytocannabinoids that can be isolated, synthesized, studied, and used for human and non-human (organic and non-organic) purposes. Naturally occurring phytocannabinoids as well as other derivations of cannabinoids (i.e. yeast derived) interact with naturally occurring human and non-human cannabinoid receptors. Known human receptors include CB-1 and CB-2, although many more human and non-human receptors are expected to be found. The most well known phytocannabinoids include delta-9-tetrahydrocannabinol (delta-9-THC), delta-8-tetrahydrocannabinol (delta-8-THC), cannabidiol (CBD), cannabigerol (CBG), cannabichromene (CBC), cannabinol (CBN), cannabicitran/cannabitriol (CBT), cannabinodiol (CBND), cannabicyclol (CBL), cannabivarin (CBV), cannabielsion (CBE), tetrahydrocannabivarin (THCV), cannabidivarin (CBDV), cannabichromevarin (CBCV), cannabigerovarin (CBGV), cannabigerol monoethyl ether (CBGM), cannabidiolic acid (CBDA), and tetrahydrocannabinolic acid (THCA).

The interaction of these phytocannabinoids and other exogenous cannabinoids with the human and non-human endocannabinoid system via CB-1, CB-2 or other cannabinoid receptors, will result in both a microscopic (i.e. cellular) and macroscopic end-organ effect for the organism.

The use of cannabis for medicinal purposes is highly regulated and so, the process of obtaining cannabis, even for medicinal, therapeutic, veterinary, or research purposes, can be tedious—even for a patient (including for non-human patients), a healthcare provider, a veterinarian, and even license holders that are suppliers of cannabis products.

### SUMMARY

The various embodiments described herein generally relate to electronic medical cannabis prescription fulfillment systems (and associated methods for operating the systems).

In accordance with some embodiments, there is provided an electronic medical cannabis prescription fulfillment system. The system includes: an interface component to receive a medical cannabis prescription issued for a patient by a healthcare practitioner; a storage component to store user data associated with each user account of one or more user accounts of the prescription fulfillment system and product data associated with each medical cannabis product available via the prescription fulfillment system; and a processor operable to: identify a user account registered with the prescription fulfillment system corresponding to the patient; generate an alert to one of the patient and the healthcare practitioner when no user account corresponding to the patient is identified, otherwise, automatically verify patient data received via the medical cannabis prescription with the user data stored in association with the user account in the storage component; display at a user interface at least one portion of the product data of each medical cannabis product available for purchase; receive a first purchase order directly from the patient to purchase a first medical cannabis product from a first licensed seller; complete the first purchaser order; subsequent to completing the first purchase order, receive a second purchase order directly from the patient to purchase a second medical cannabis product from a second licensed seller different from the first licensed seller; and in response to receiving the second purchase order, automatically validate the medical cannabis prescription and complete the second purchase order when the medical cannabis prescription is validated.

In some embodiments, the processor is operable to: in response to receiving the second purchase order, determine whether the medical cannabis prescription is expired to validate the medical cannabis prescription.

In some embodiments, the processor is operable to: trigger the patient to create the user account with the prescription fulfillment system when no user account corresponding to the patient is identified.

In some embodiments, the processor is operable to: trigger the healthcare practitioner to notify the patient to create the user account with the prescription fulfillment system when no user account corresponding to the patient is identified.

In some embodiments, the processor is operable to: receive a new account request from the patient to create the user account; and in response to receiving the new account request, display at the user interface one or more account creation user interfaces for receiving at least some of the user data.

In some embodiments, the processor is operable to: receive a portion of the user data from at least one of the healthcare practitioner and the medical cannabis prescription.

In some embodiments, the processor is operable to: store the received user data in association with the user account created for the patient in the storage component.

In some embodiments, the processor is operable to: compare the patient data with the stored user data to identify one or more discrepancies; and automatically notify the healthcare practitioner of the one or more discrepancies when identified.

In some embodiments, the processor is operable to: detect abnormal activity associated with at least one of the healthcare practitioner and the user account based on the received medical cannabis prescription.

In some embodiments, the processor is operable to: automatically order the selection of medical cannabis products for the patient based on at least one of the user data, prescription data received from the medical cannabis prescription, and product data associated with each medical cannabis product.

In some embodiments, the processor is operable to automatically order the selection of medical cannabis products for the patient based on a medical condition identified in the medical cannabis prescription.

In some embodiments, the processor is operable to: determine a historical medical cannabis usage from the user data stored in the storage component accessible via a network; and automatically order the selection of medical cannabis products for the patient based on the historical medical cannabis usage.

In some embodiments, the processor is operable to: determine a medical cannabis usage preference from the user data stored in a storage component accessible via a network; and automatically order the selection of medical cannabis products for the patient based on the medical cannabis usage preference.

In some embodiments, the processor is operable to: display the at least one portion of the product data of one or more cannabis products produced by two or more licensed sellers.

In accordance with some embodiments, there is provided a computer-implemented method for operating an electronic medical cannabis prescription fulfillment system. The method includes: receiving a medical cannabis prescription issued for a patient by a healthcare practitioner; and operating a processor to: identify a user account registered with the prescription fulfillment system corresponding to the patient; generate an alert to one of the patient and the healthcare practitioner when no user account corresponding to the patient is identified, otherwise, automatically verify patient data from the medical cannabis prescription with the user data stored in association with the user account in a storage component; display at a user interface at least one portion of the product data of each medical cannabis product available for purchase; receive a first purchase order directly from the patient to purchase a first medical cannabis product from a first licensed seller; complete the first purchaser order; subsequent to completing the first purchase order, receive a second purchase order directly from the patient to purchase a second medical cannabis product from a second licensed seller different from the first licensed seller; and in response to receiving the second purchase order, automatically validate the medical cannabis prescription, and complete the second purchase order, when the medical cannabis prescription is validated.

In some embodiments, the method includes operating the processor to automatically validate the medical cannabis prescription by determining whether the medical cannabis prescription is expired.

In some embodiments, the method includes operating the processor to generate the alert to one of the patient and the healthcare practitioner when no user account corresponding to the patient is identified includes: triggering the patient to create the user account with the prescription fulfillment system.

In some embodiments, the method includes operating the processor to generate the alert to one of the patient and the healthcare practitioner when no user account corresponding to the patient is identified includes: triggering the healthcare practitioner to notify the patient to create the user account with the prescription fulfillment system.

In some embodiments, the method includes operating the processor to generate the alert to one of the patient and the healthcare practitioner when no user account corresponding to the patient is identified includes: receiving a new account request from the patient to create the user account; and in response to receiving the new account request, displaying at the user interface one or more account creation user interfaces for receiving at least some of the user data.

In some embodiments, the method includes operating the processor to receive a portion of the user data from at least one of the healthcare practitioner and the medical cannabis prescription.

In some embodiments, the method includes operating the processor to: store the received user data in association with the user account created for the patient in the storage component.

In some embodiments, the method includes operating the processor to automatically verify the patent data includes: comparing the patient data with the stored user data to identify one or more discrepancies; and automatically notifying the healthcare practitioner of the one or more discrepancies when identified.

In some embodiments, the method includes operating the processor to: detect abnormal activity associated with at least one of the healthcare practitioner and the user account based on the received medical cannabis prescription.

In some embodiments, the method includes operating the processor to display at least one portion of the product data of each medical cannabis product available for purchase to the patient by: automatically ordering the selection of medical cannabis products for the patient based on at least one of the user data, prescription data received from the medical cannabis prescription, and product data associated with each medical cannabis product.

In some embodiments, the method includes operating the processor to automatically order the selection of medical cannabis products for the patient by automatically ordering the selection of medical cannabis products for the patient based on a medical condition identified in the medical cannabis prescription.

In some embodiments, the method includes operating the processor to automatically order the selection of medical cannabis products for the patient by determining a historical medical cannabis usage from the user data stored in the storage component accessible via a network; and automatically ordering the selection of medical cannabis products for the patient based on the historical medical cannabis usage.

In some embodiments, the method includes operating the processor to automatically order the selection of medical cannabis products for the patient by determining a medical cannabis usage preference from the user data stored in a storage component accessible via a network; and automatically ordering the selection of medical cannabis products for the patient based on the medical cannabis usage preference.

In some embodiments, the method includes operating the processor to display the at least one portion of the product data of each medical cannabis product available for purchase to the patient by displaying the at least one portion of the product data of one or more cannabis products produced by two or more licensed sellers.

In some embodiments, the healthcare practitioner is selected from a group consisting of a doctor and a nurse practitioner.

### BRIEF DESCRIPTION OF THE DRAWINGS

Several embodiments will now be described in detail with reference to the drawings, in which:

FIG. 1 is a block diagram of components interacting with an electronic medical cannabis prescription fulfillment system in accordance with an example embodiment;

FIG. 2 is a flowchart of an example embodiment of various methods of operating an example electronic medical cannabis prescription fulfillment system;

FIG. 3 shows an example user interface illustrating an example medical cannabis prescription;

FIG. 4 shows an example user interface displaying some medical cannabis products available via the electronic medical cannabis prescription fulfillment system disclosed herein, in accordance with an example embodiment;

FIG. 5 shows another example user interface displaying some medical cannabis products available via the electronic medical cannabis prescription fulfillment system disclosed herein, in accordance with an example embodiment; and

FIG. 6 shows an example user interface illustrating example purchase orders.

The drawings, described below, are provided for purposes of illustration, and not of limitation, of the aspects and features of various examples of embodiments described herein. For simplicity and clarity of illustration, elements shown in the drawings have not necessarily been drawn to scale. The dimensions of some of the elements may be exaggerated relative to other elements for clarity. It will be appreciated that for simplicity and clarity of illustration, where considered appropriate, reference numerals may be repeated among the drawings to indicate corresponding or analogous elements or steps.

### DESCRIPTION OF EXAMPLE EMBODIMENTS

Use of cannabis for medicinal purposes, or medical cannabis, has grown in recent years. Many countries, such as Australia, Canada, Chile, Colombia, Germany, Israel, Jamaica, Norway, Thailand and the United Kingdom have legalized medical cannabis. In the United States, medical cannabis has been legalized in many states but continues to be prohibited at the federal level. Although medical cannabis is now more common, the use, sale and purchase of medical cannabis continues to be highly regulated.

In Canada, for example, once a patient (human or non-human) is issued a prescription for medical cannabis by a healthcare practitioner, the patient needs to register the prescription with a licensed seller of cannabis. The healthcare practitioner can include a veterinarian for non-human patients, for example. The licensed seller can include any entity licensed by the applicable laws to sell cannabis, including, but not limited to cannabis producers, cultivators and/or processors. Some licensed sellers may not be a cannabis producer, cultivator, and/or processor. The patient is then restricted to purchasing products from that licensed seller. This can be frustrating for patients as the medical effects of cannabis can vary with the exogenous cannabinoid composition from each strain grown by each producer, and patients may prefer to have access to diverse cannabinoid profiles and/or continuous access to products with comparable cannabinoid profiles from multiple different producers.

To purchase products from a different licensed seller, the patient is required to obtain a new prescription from their healthcare practitioner, cancel their registration with the current licensed seller, and register the new prescription with the new licensed seller. This process is mostly intended to prevent fraudulent use of the medical cannabis prescription—for example, to prevent patients from accessing more than prescribed limits. This is a tedious process, especially for the patient, who can be left with gaps in treatment, and in conditions like seizures or epileptiform disorders, there may be fatal consequences from the loss of access to cannabinoid treatment(s).

With the disclosed electronic medical cannabis prescription fulfillment system, patients issued a prescription for medical cannabis can purchase cannabis products from a variety of licensed sellers registered with the electronic medical cannabis prescription fulfillment system. The electronic medical cannabis prescription fulfillment system electronically receives the medical cannabis prescription from a healthcare practitioner. The electronic medical cannabis prescription fulfillment system then associates the medical cannabis prescription to a user account. When the user account is located, or created, the electronic medical cannabis prescription fulfillment system can then verify the patient data provided in the medical cannabis prescription against the user data stored in association with the user account.

Via the electronic medical cannabis prescription fulfillment system, the patient is able to select from cannabis products offered by different licensed sellers. For example, when the patient wants to purchase a cannabis product from a different licensed seller, the electronic medical cannabis prescription fulfillment system automatically validates the stored medical cannabis prescription before completing the purchase order with the new licensed seller. The electronic medical cannabis prescription fulfillment system may also automatically cancel the registration of that patient's medical cannabis prescription with the prior licensed seller, and automatically register the medical cannabis prescription with the new licensed seller. The disclosed electronic medical cannabis prescription fulfillment system can automatically verify and manage the medical cannabis prescription for the patient.

The electronic medical cannabis prescription fulfillment system can enable the patient to select from cannabis products offered by different licensed sellers. The electronic medical cannabis prescription fulfillment system can also operate to validate the medical cannabis prescription and determine whether the requested purchase order exceeds prescribed limits—as regulated by the medical cannabis prescription and/or by applicable laws. For example, when the electronic medical cannabis prescription fulfillment system receives a purchase order, the electronic medical cannabis prescription fulfillment system can validate the stored medical cannabis prescription and also determine whether the purchase order would exceed the prescribed limits based on the historical purchases associated with that user account.

In some embodiments, the electronic medical cannabis prescription fulfillment system can operate to display only amounts of cannabis products that are can be purchased by that patient, as limited by the prescribed limits. For example, the electronic medical cannabis prescription fulfillment system can determine an available quantity of cannabis products that the patient can purchase based on a prescribed limit for a period of time based on past purchase orders associated that user account. The electronic medical cannabis prescription fulfillment system can then restrict the purchase order to that available quantity—by restricting selection of the quantity only to the available quantity and/or automatically reducing the purchase order to the available quantity. The electronic medical cannabis prescription fulfillment system can also indicate via the display when the patient can purchase additional quantities, in accordance with the prescribed limits. The electronic medical cannabis prescription fulfillment system can also identify any fixed-volume packages of the cannabis orders that can cause the purchase order to exceed the prescription and/or authorized amount as prescribed by the applicable laws. The electronic medical cannabis prescription fulfillment system can then remove those fixed-volume packages from being displayed and/or indicate that those fixed volume packages cannot be ordered.

The electronic medical cannabis prescription fulfillment system can also operate to synchronize inventory of the licensed sellers according to the purchase orders.

Reference is first made to FIG. 1, which illustrates a block diagram 100 of components interacting with an electronic medical cannabis prescription fulfillment system 110.

The electronic medical cannabis prescription fulfillment system 110 includes a processor 112, an interface component 114 and a storage component 116. As shown in FIG. 1, the electronic medical cannabis prescription fulfillment system 110 can be in communication with an external storage component 120 and a computing device 130 via a network 150. Although the electronic medical cannabis prescription fulfillment system 110 is shown as one component in FIG. 1, in some embodiments, the electronic medical cannabis prescription fulfillment system 110 can be provided with one or more servers distributed over a wide geographic area and connected via the network 150.

The processor 112 may be any suitable processor, controller or digital signal processor that provides sufficient processing power depending on the configuration, purposes and requirements of the electronic medical cannabis prescription fulfillment system 110. In some embodiments, the processor 112 can include more than one processor with each processor being configured to perform different dedicated tasks.

The processor 112 controls the operation of the electronic medical cannabis prescription fulfillment system 110. For example, the processor 112 can verify a received medical cannabis prescription and/or can locate a user account associated with a patient issued the medical cannabis prescription. The processor 112 can, in some embodiments, operate to manage purchase orders to ensure the purchase orders do not result in excess of prescribed limits.

The interface component 114 may be any interface that enables the electronic medical cannabis prescription fulfillment system 110 to communicate with other devices and systems. In some embodiments, the interface component 114 can include at least one of a serial port, a parallel port or a USB port. The interface component 114 may also include at least one of an Internet, Local Area Network (LAN), Ethernet, Firewire, modem or digital subscriber line connection. Various combinations of these elements may be incorporated within the interface component 114.

For example, the interface component 114 may receive input from various input devices, such as a mouse, a keyboard, a touch screen, a thumbwheel, a track-pad, a track-ball, a card-reader, voice recognition software and the like depending on the requirements and implementation of the electronic medical cannabis prescription fulfillment system 110.

The storage component 116 can include RAM, ROM, one or more hard drives, one or more flash drives or some other suitable data storage elements such as disk drives, etc. For example, the storage component 116 can include a memory on which one or more databases or file system(s) are stored. The database(s) can, but is not limited to, store information related to the users, cannabis products available via the electronic medical cannabis prescription fulfillment system 110, and licensed sellers. For example, the storage component 116 can store user data, such as, but not limited to, personal information (e.g., name, sex, age, birthdate, contact information, health card number, healthcare practitioner information, ethnicity, genetic information, cytochrome p450 metabolism information, electronic laboratory information, personal cannabis use history, medical co-morbidities, active medications, supplements, vitamins, herbal or ancient medicine usage, allergies, smoking status, alcohol co-ingestion status, occupation, family history, risk factors including mental health status, medical diagnosis, symptoms, frequency of symptoms, severity of symptoms, diagnostic test results, insurance policy and reimbursement information, hobbies, activities, personal interests, etc.), login credentials, active and past purchase orders, preferred methods of payment, preferred addresses and shipping details, current and past shipping and tracking information, past refunds and returns, medical cannabis prescription(s) details including amounts remaining in the prescription per authorized period (e.g., monthly, weekly, etc.), past cannabis prescription fulfillment system usage, preferences and projections, pre-order preferences, personal product reviews, symptom response and therapeutic and medical reviews (including standardized validated and non-validated medical and research questionnaires), symptom and diagnostic trajectories, information from personal electronic devices, information from other electronic devices for health data measurement and recording, etc.

The storage component 116 can also store product data for each cannabis product available via the electronic medical cannabis prescription fulfillment system 110, such as, but not limited to, plant genetics, authentication elements including plant distributed ledger systems (e.g., based on blockchain technology), strain certificate of analysis data, product specific branding and marketing elements, brand chemical composition, medical effect(s), price (e.g., discounted prices after all inducements, such as via insurance policy coverage), remaining inventory, categorization elements for the purposes of informed decision making such as time of use (e.g., morning, daytime, evening, sleep), and filtration elements such as medical effects including medical diagnosis or symptoms (e.g., to filter the phytocannabinoid profile that would yield maximum real-world data guided benefit for the selected diagnosis or symptom). In some embodiments, the processor 112 can operate to verify the product data for each cannabis products. For example, the processor 112 can verify a composition of the exogenous cannabinoids and whether the composition is compliant with the applicable laws. The processor 112 can verify the composition against an electronic certificate of analysis provided by an authorized testing facility. The electronic certificate of analysis may be stored in the storage component 116 or via the network 150.

The storage component 116 can also store data related to limited stock items and out-of-stock items, past cannabis prescription fulfillment system usage, preferences, and projections for a product, product reviews, product returns, product refunds, product complaints, product recalls, recall status, symptom response and therapeutic and medical reviews for a product (including standardized validated and non-validated medical and research questionnaires) and licensed seller data (such as, but not limited to, registration information, production details, unique production or product elements, industry differentiators, currently listed products including all packaging, labels, and images, past cultivation data, past processing data, past testing data, past research data, past reports and analytics, past returns, past refunds, past complaints, past recalls, past contaminations, sales prices per gram, sales prices per mg, sales prices per mg of cannabinoid(s), total sales, total sales per product, total sales per product relative to inventory, total sales taxes, total fees, total shipping costs, total costs, gross revenue, net revenue, revenue per product, revenue per strain, revenue per mg of cannabinoid, sales projections, projected inventory requirements, pre-orders, aggregate data from other licensed sellers, past cannabis prescription fulfillment system usage, sales, preferences, and projections per product, product reviews, symptom response and therapeutic and medical reviews per product (including standardized validated and non-validated medical and research questionnaires, trajectories), unlisted products etc.).

In some embodiments, an application can be stored on the storage component 116, and executable by the processor 112.

Similar to the storage component 116, the external storage component 120 can include RAM, ROM, one or more hard drives, one or more flash drives or some other suitable data storage elements such as disk drives, etc. The external storage component 120 can include a memory on which one or more databases or file system(s) are stored. Although only one external storage component 120 is shown, there may be multiple external storage components 120 distributed over a wide geographic area and connected via the network 150.

The external storage component 120 can be accessed by the electronic medical cannabis prescription fulfillment system 110 via the network 150. The external storage component 120 can act as a back-up storage component to the storage component 116 and/or store at least some of the data related to the users, cannabis products available via the electronic medical cannabis prescription fulfillment system 110, licensed sellers, and/or ongoing data received from various users of the electronic medical cannabis prescription fulfillment system 110. In some embodiments, the external storage component 120 can store data that is not as frequently used by the electronic medical cannabis prescription fulfillment system 110, or larger size data.

Each of the processor 112, the interface component 114 and the storage component 116 may be combined into a fewer number of components or may be separated into further components. The processor 112, the interface component 114 and the storage component 116 may be implemented in software or hardware, or a combination of software and hardware.

The computing device 130 may be any networked device operable to connect to the network 150. A networked device is a device capable of communicating with other devices through a network such as the network 150. A network device may couple to the network 150 through a wired or wireless connection.

Although only one computing device 130 is shown in FIG. 1, there may be multiple computing devices 130 in communication with the electronic medical cannabis prescription fulfillment system 110 via the network 150. The computing devices 130 can be distributed over a wide geographic area. For example, a healthcare clinic, a non-healthcare clinic, a healthcare practitioner, a pharmacy, a hospital, or any authorized licensed or non-licensed individual or entity under applicable law can use a computing device 130 to electronically transfer a medical cannabis prescription to the electronic medical cannabis prescription fulfillment system 110 via the network 150. In another example, a patient can use a computing device 130 to access the electronic medical cannabis prescription fulfillment system 110 for creating a user account and/or submitting a purchase order. In another example, a licensed seller can use the computing device 130 to manage the inventory of products available for purchase via the electronic medical cannabis prescription fulfillment system 110. The licensed seller can also buy, sell, and/or transfer products to another licensed seller located in another geographic region.

The computing devices 130 may include at least a processor and memory, and may be an electronic tablet device, a personal computer, workstation, server, portable computer, mobile device, personal digital assistant, laptop, smart phone, WAP phone, an interactive television, video display terminals, gaming consoles, and portable electronic devices or any combination of these. In some embodiments, the computing device 130 may be a laptop or a smartphone device equipped with a network adapter for connecting to the Internet.

The network 150 may be any network capable of carrying data, including the Internet, Ethernet, electronic facsimile, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these, capable of interfacing with, and enabling communication between the electronic medical cannabis prescription fulfillment system 110, the computing device 130 and/or the external storage component 120.

Referring now to FIG. 2, which is a flowchart diagram illustrating an example method 200 of operating the electronic medical cannabis prescription fulfillment system 110 disclosed herein. To illustrate the method 200, reference will be made to FIGS. 3 to 6.

At 210, the electronic medical cannabis prescription fulfillment system 110 receives a medical cannabis prescription issued for a patient by a healthcare practitioner.

The healthcare practitioner can include any individual authorized to prescribe medical cannabis to their patients. The healthcare practitioner can include a doctor and/or a nurse practitioner. The electronic medical cannabis prescription fulfillment system 110 can receive the medical cannabis prescription via a computing device 130 associated with the healthcare practitioner. For example, the electronic medical cannabis prescription fulfillment system 110 can receive the medical cannabis prescription directly from a computer workstation operated by the healthcare practitioner at any location of practice such as a virtual clinic, clinic or hospital, or from a transmission device operated by a personnel employed by a virtual clinic, clinic, pharmacy, hospital or healthcare practitioner in which the healthcare practitioner practices or is associated.

Generally shown in FIG. 3, at 300, is an example user interface 310 illustrating an example medical cannabis prescription 312 for a user (“John Smith”) associated with a user account 320. The example medical cannabis prescription 312 includes patient data (e.g., a patient name 332 and a cannabis registration identifier 334), practitioner data (e.g., a practitioner name 336 and a practitioner registration identifier 338), a medical issue or condition field 340 (e.g., medical diagnosis or symptom), a prescription date 330, a prescription expiry date 344 and prescribed dosage 342. A note field 350 is also included in the example medical cannabis prescription 312. The medical cannabis prescription 312 shown herein is for illustrative purposes. Fewer and/or additional data fields may be included, such as, for example, restrictions to the prescription (e.g., maximum % THC or up-titration restrictions) that can be monitored and regulated by the electronic medical cannabis prescription fulfillment system 110.

When the electronic medical cannabis prescription fulfillment system 110 receives the medical cannabis prescription 312 via the network 150, the electronic medical cannabis prescription fulfillment system 110 can retrieve patient data (e.g., patient name 332 and/or cannabis registration identifier 334) from the medical cannabis prescription 312 to identify a corresponding user account and associated data for the patient 332. The electronic medical cannabis prescription fulfillment system 110 can also store the received medical cannabis prescription 312 in the storage component 116.

At 220, the electronic medical cannabis prescription fulfillment system 110 operates the processor 112 to identify a user account 320 corresponding to the patient 332.

When the processor 112 identifies the user account 320 (at 230), the electronic medical cannabis prescription fulfillment system 110 operates the processor 112 to automatically verify the patient data received via the medical cannabis prescription 312 with the user data stored in association with the user account 320 (at 232).

To verify the patient data, the electronic medical cannabis prescription fulfillment system 110 can compare the patient data received via the medical cannabis prescription 312 with the user data stored in the storage component 116 and/or the external storage component 120. For example, with respect to the medical cannabis prescription 312 shown in FIG. 3, the electronic medical cannabis prescription fulfillment system 110 can retrieve the patient name 332 and the cannabis registration identifier 334, and can locate a corresponding user account based on the patient name 332 and the cannabis registration identifier 334. The processor 112 successfully located the user account 320 when at least the patient name 332 matches the stored user name, and the cannabis registration identifier 334 matches the stored registration identifier.

In some embodiments, the electronic medical cannabis prescription fulfillment system 110 may identify the user account 320 when the cannabis registration identifier 334 matches with the stored registration identifier.

In some embodiments, the electronic medical cannabis prescription fulfillment system 110 may identify the user account 320 only after additional information is verified. For example, the electronic medical cannabis prescription fulfillment system 110 may require additional fields, such as but not limited to, the medical issue field 340 and/or practitioner data (the practitioner name 336 and/or the practitioner registration identifier 338) to match the corresponding stored fields before identifying the user account 320. In some embodiments, the electronic medical cannabis prescription fulfillment system 110 may generate a notification to the healthcare practitioner 336 and/or the patient 332 requesting further information before identifying any user accounts 320. For example, the electronic medical cannabis prescription fulfillment system 110 may generate the notification following one or more unsuccessful attempts to access identify the user account 320.

The enhanced verification process may be required by user preferences as defined in the user account and/or preferences of the practitioner. In some cases, the electronic medical cannabis prescription fulfillment system 110 may be triggered to require additional verification data when greater security threats and/or fraudulent activities are detected at the system 110 and/or within the general medical cannabis domain including, but not limited to, multiple user accounts 320 with the same patient 332, the same shipping address in user account 320 or the same payment details, issued by one or more healthcare practitioners 336.

In some embodiments, the electronic medical cannabis prescription fulfillment system 110 may be triggered to require additional verification data when more than one password with the same username for user account 320 is inputted using a computing device 130 via the network 150. For example, after predefined number of incorrect login attempts, the electronic medical cannabis prescription fulfillment system 110 can lock the user account 320 and prevent access to the electronic medical cannabis prescription fulfillment system 110. The electronic medical cannabis prescription fulfillment system 110 can then notify the patient 332 and/or the health practitioner 336 via the network 150 of the login attempts and require a password reset for the user account 320. The electronic medical cannabis prescription fulfillment system 110 may also be triggered to require additional verification data if matching payment information stored in the storage component 116 is entered by another user account 320 or is shipped to an address that does not match the shipping address stored with that payment information for that user account 320 in the storage component 116.

In some embodiments, the electronic medical cannabis prescription fulfillment system 110 may be triggered to require additional verification data when it receives a request order from a user account 320 that exceeds the prescribed dosage 342, limits imposed by the health practitioner 336 and/or beyond the prescription expiry date 344. In such cases, the electronic medical cannabis prescription fulfillment system 110 can operate the processor 112 to automatically notify the patient 332, and if authorized by the patient 332, the healthcare practitioner 336, associated with user account 320, to reassess or renew the prescription 312. The electronic medical cannabis prescription fulfillment system 110 may be triggered to require additional verification data if the shipping address does not comply with applicable law, for storage in the storage component 116, prior to denying the patient 332 associated with the user account 320 of the purchase order. In such cases, the electronic medical cannabis prescription fulfillment system 110 can operate the processor 112 to automatically notify the patient 332 and/or the healthcare practitioner 336, that an attempt to ship outside the jurisdictions of the applicable law has been attempted by the patient 336 or another operator of the user account 320.

In some embodiments, after identifying the user account 320 for the patient 332, the electronic medical cannabis prescription fulfillment system 110 can, compare the patient data (e.g., 332, 334) with the stored user data to identify any discrepancies. For example, the patient name 332 in the medical cannabis prescription 312 may not completely match the user name stored in the storage component 116, 120. When a discrepancy is identified, the electronic medical cannabis prescription fulfillment system 110 can operate the processor 112 to automatically notify the healthcare practitioner 336 of the discrepancy.

The verification process conducted by the processor 112 can involve, in some embodiments, detecting abnormal activities associated with the healthcare practitioner 336 (and/or associated healthcare clinic) including the source issuing the medical cannabis prescription 312, and/or the user account 320. For example, the processor 112 can be triggered to monitor the medical cannabis prescriptions 312 issued by a healthcare practitioner 336 when a weekly prescription threshold has been exceeded for healthcare practitioner 336, when the medical cannabis prescription 312 is received outside the normal operating hours of the healthcare practitioner 336, or when variations beyond predicted norms are detected by the processor 112 for the user account associated with healthcare practitioner 336. The processor 112 can also be triggered to monitor a user account 320 when multiple medical cannabis prescriptions 312 have been issued for the patient 332, or if the shipping address or payment information associated with user account 320 is associated with multiple medical prescriptions 312 issued by healthcare practitioner 336. The processor 112 can also be triggered to monitor a user account 320 when the medical cannabis prescriptions 312 are being issued by healthcare practitioners 336 located outside a maximum surrounding area of the residence of the patient 332 or authorized under applicable law. The processor 112 can be triggered if multiple user accounts for a healthcare practitioner 336 are created with the same practitioner name 336 or practitioner license number 338. In such cases the processor 112 can store in the storage component 116 data related to purchase orders from user account(s) 320 including shipping information and payment information for reporting to relevant authorities, such as licensing officials.

In some embodiments, the processor 112 can be triggered to verify the authenticity of the medical cannabis prescription 312 itself. The processor 112 can verify the healthcare practitioner name 336 and license number 338 stored in the storage component 116 to verify the authenticity of the healthcare practitioner. The processor 112 can also determine any limitations or restrictions associated with the license of the healthcare practitioner related to the practice of medicine, nursing and/or medical cannabis. The processor 112 can also review the fields of the prescription 312 to identify errors and/or omissions, and for compliance with applicable laws. The processor 112 can also review the prescription 312 to identify any variations in the electronic signature of the healthcare practitioner 336. The processor 112 can review the prescribed dosage 342 to ensure compliance with the allowed dosage under applicable law. The processor 112 can verify that the prescription expiry 344 does not violate the maximum valid period for a prescription under applicable law. The processor 112 can verify the postal code or zip code associated with the prescription 312 for compliance with applicable law for access to medical cannabis including but not limited to online purchase and mail-order delivery. The processor 112 can also ensure that there is no duplicate account for the same patient 332.

When the processor 112 does not identify the user account 320 (at 230), the electronic medical cannabis prescription fulfillment system 110 operates the processor 112 to generate an alert to one of the patient 332 and the healthcare practitioner 336 to trigger creation of the user account 320 (at 234). After the user account 320 is created, the processor can proceed to 220 to identify the user account 320 for the patient 332.

In some embodiments, the electronic medical cannabis prescription fulfillment system 110 can operate the processor 112 to trigger the patient 332 to create the user account 320. The processor 112 can generate the alert to include an electronic link to a registration interface, and transmit the alert via the network 150 to the patient 332 and/or the healthcare practitioner 336. When the alert is sent to the healthcare practitioner 336, the healthcare practitioner 336 can forward the alert to the patient 332 and/or create the user account 320 with the patient 332.

When the electronic link is selected by the patient 332 and/or the healthcare practitioner 336 on behalf of the patient 332, the processor 112 receives a new account request. In response to receiving the new account request, the processor 112 displays an account creation user interface for receiving at least some of the user data to be stored at the storage component 116, 120. In some embodiments, the patient 332 and/or the healthcare practitioner 336 can be directed to the account creation user interface via other avenues, such as by navigating directly to a webpage operated by the electronic medical cannabis prescription fulfillment system 110. The processor 112 can display the account creation user interface via the interface component 114, for example, at a user display of the computing device 130 operated by the patient 332 and/or the healthcare practitioner 336. Via the account creation user interface, the electronic medical cannabis prescription fulfillment system 110 can receive various user data from the patient 332, such as but not limited to, personal information, medical information (including laboratory results), past cannabis usage, cannabis preferences, product reviews, symptom and medical outcomes reviews, health and disease information from electronic devices used by patient 332, etc.

In some embodiments, the electronic medical cannabis prescription fulfillment system 110 can receive a portion of the user data from other sources, such as the healthcare practitioner 336 and/or the medical cannabis prescription 312.

The electronic medical cannabis prescription fulfillment system 110 can store the received user data in association with the user account 320 created for the patient 332 in the storage component 116, 120.

At 240, the electronic medical cannabis prescription fulfillment system 110 operates the processor 112 to display at a user interface at least one portion of the product data of each cannabis product 420 available for purchase.

Reference will now be made to FIG. 4, which is an example user interface 410 (as shown generally at 400) displaying some cannabis products 420a, 420b, 420c available via the electronic medical cannabis prescription fulfillment system 110.

Various product data is shown for each cannabis product 420. For example, for each cannabis product 420a, 420b and 420c, the illustrated product data includes a respective product name 422a, 422b, 422c (“ABC's Dream”, “High Shelf”, “X-Flower”, respectively), a product price, a licensed seller name 424a, 424b, 424c (“ABC Crops”, “Top Grower”, “XYZ Grow”, respectively), associated benefits, and additional notes. A quantity selection control 430a, 430b, 430c is also provided for each illustrated cannabis product 420a, 420b and 420c, respectively.

The illustrated product data is only for exposition purposes. Fewer or more product data can be provided. The amount of data illustrated may vary with the user account 320 and/or the associated licensed seller 424, for example, including available supplies or listed products, and restrictions or limits prescribed by the healthcare practitioner 332 in the prescription notes 350, for example. Also, although three cannabis products 420 are illustrated in FIG. 4, it will be understood that fewer or more cannabis products 420 could be illustrated in the user interface 410. It is possible that the cannabis products 420 of fewer or more licensed seller can be illustrated.

The cannabis products 420 illustrated can vary with the user data associated with the user account 320, in some embodiments. For example, the user can exclude the cannabis products 420 from certain licensed sellers 424 from being displayed or limit the user interface 410 to display only the cannabis products 420 from a specific licensed seller 424 to comply with prescribed limits of 310 or user account 320 preferences.

When the cannabis products 420 are displayed on the user interface 410, the electronic medical cannabis prescription fulfillment system 110 can operate the processor 112 to automatically order the cannabis products 420 for the patient 332 based on the user data, prescription data received from the medical cannabis prescription 312, and/or product data associated with each cannabis product 420 in accordance with the user account's 320 preferences. For example, the processor 112 can automatically select the cannabis products 420 based on the medical condition or issue (e.g., nausea) 340 identified in the medical cannabis prescription 312. The processor 112 can, in some embodiments, order the cannabis products 420 according to the medical condition or issue 340, instead of or in addition to selecting a subset of the cannabis products 420.

FIG. 5 shows, at 500 generally, an example user interface 510 displaying some cannabis products 420a, 420d, 420e. Each cannabis product 420a, 420d, 420e is associated with the benefit of relieving nausea, which is the issue noted in the medical issue field 340 of the medical cannabis prescription 312.

In some embodiments, the electronic medical cannabis prescription fulfillment system 110 can operate the processor 112 to determine a historical medical cannabis usage from the user data. The electronic medical cannabis prescription fulfillment system 110 may generate projections based on the historical medical cannabis usage, in some examples. The processor 112 can then be operated to automatically select the displayed cannabis products 420 based on the historical medical cannabis usage and/or projections. The processor 112 can, in some embodiments, order the cannabis products 420 according to the historical medical cannabis usage and data projections, instead of or in addition to selecting a subset of the cannabis products 420. For example, from the note field 350 of the medical cannabis prescription 312, the processor 112 can determine that the patient 332 has minimal prior medical cannabis usage. The processor 112 can then select the cannabis products 420 recommended for beginner users, such as cannabis products 420a, 420e. The processor 112 can also prioritize the cannabis products 420 based on the recommended user level factoring in elements such as, but not limited to, past cannabis exposure including methods of consumption, potency of products consumed, quantities consumed, frequency of consumption, last exposure or consumption, physiological tolerance, physiological dependence, risk factors for side effects, risk factors for abuse, optimal dose (e.g., based on age, sex, weight, genetics etc.), drug-drug interactions, bioavailability, variations in metabolism based on the cytochrome p450 profile of the user, potential for toxicity or an adverse reaction based on the product profile, etc.

In some embodiments, the processor 112 can also automatically select the medical cannabis products 420 based on, but not limited to, supplier inventory, shipping carrier rates, disease and symptom temporal fluctuations, seasonal/monthly/weekly/daily variations, past exogenous cannabinoid composition consumed including method, route of delivery, dose (cannabinoid composition), frequency, and both aggregate and user-specific retrospective product reviews including standardized feedback in the form of disease or symptom specific validated medical questionnaires, for example.

The electronic medical cannabis prescription fulfillment system 110 can operate the processor 112 to determine a medical cannabis usage preference from the user data. The processor 112 can then automatically select the cannabis products 420 based on the determined medical cannabis usage preference. The processor 112 can, in some embodiments, the processor 112 can order the cannabis products 420 according to the medical cannabis usage preference, instead of or in addition to selecting a subset of the cannabis products 420.

For example, with reference to the user account 320, the user can indicate a preference for a type of cannabis product 420, a strength of the cannabis product 420, the cannabis products 420 grown by a specific licensed seller 424, a price for the cannabis product 420, a specific cannabinoid ratio in a particular form (e.g., oil) of medical cannabis, and/or another factor associated with the cannabis product 420 or licensed seller 424. Based on the medical cannabis usage preference data defined in the user account, the processor 112 can select the cannabis products 420 to satisfy the medical cannabis usage preference.

The processor 112 can be operated to automatically select the cannabis products 420 based on one or more factors. For example, the processor 112 can automatically select the cannabis products 420 based on the medical condition or issue 340 and historical medical cannabis usage.

In some embodiments, the processor 112 can determine a geographic location of the user when accessing the user account 320. The processor 112 can determine the geographic location of the user via various methods, such as via an Internet Protocol (IP) associated with the computing device 130 with which the login request was made. The processor 112 can then display cannabis products available to be delivered to that geographic location. In some embodiments, when the geographic location is determined to be different from a billing address and/or home address of the user, the processor 112 may request information on the intended shipping address from the user and then display cannabis products available to be delivered to that shipping address. For example, the cannabis products may be available only from the licensed products local to that shipping address.

At 250, the electronic medical cannabis prescription fulfillment system 110 operates the processor 112 to receive a first purchase order 630a directly from the patient 332 to purchase a first cannabis product 420a from a first licensed seller 424a.

FIG. 6 shows an example user interface 610, generally at 600, that illustrates example purchase orders 630a, 630b. For each purchase order 630a, 630b, order information, such as purchase date 620a, 620b, purchased cannabis product names 420a, 420d (“ABC's Dreams” and “Dream On”, respectively), licensed sellers of the purchased cannabis products 424a, 424d (“ABC Crops” and “Flower Boss”, respectively), purchased quantity 622a, 622b and order status 624a, 624b are provided.

The electronic medical cannabis prescription fulfillment system 110 operates the processor 112 to complete the first purchase order 630a (at 260). As shown at 624a in FIG. 6, the processor 112 completed the first purchase order 630a. The first purchase order 630a was for the first cannabis product 420a produced by the first licensed seller 424a.

In some embodiments, to complete the first purchase order 630a, the processor 112 can be operated to register the medical cannabis prescription 312 with the first licensed seller 424a. When registering the medical cannabis prescription 312 with the first licensed seller 424a, the processor 112 can be operated to determine the registration requirements associated with the first licensed seller. Each licensed seller 424 can have different registration requirements. When additional information is required to complete the registration, the processor 112 can be operated to retrieve the additional information from the storage component 116, 120 and/or obtain the additional information from the patient 332 and/or healthcare practitioner 336 via the network 150.

At 270, subsequent to completing the first purchase order 630a, the electronic medical cannabis prescription fulfillment system 110 operates the processor 112 to receive a second purchase order 630b directly from the patient 332 to purchase a second cannabis product 420d from a second licensed seller 424d different from the first licensed seller 424a.

The electronic medical cannabis prescription fulfillment system 110 can receive multiple purchase orders 630a, 630b from the user account 320 from different computing devices 130—it is possible that the orders 630a, 630b are received from different computing devices 130 at substantially the same time. The electronic medical cannabis prescription fulfillment system 110 can provide the licensed sellers 424a, 424d, respectively, of the purchase orders 630a, 630b, and the licensed sellers 424a, 424d can fulfill the purchase orders 630a, 630b via drop-shipping, for example.

In response to receiving the second purchase order 630b, at 280, the electronic medical cannabis prescription fulfillment system 110 operates the processor 112 to automatically validate the medical cannabis prescription 312 and complete the second purchase order 630b when the medical cannabis prescription 312 is validated. The electronic medical cannabis prescription fulfillment system 110 can validate the medical cannabis prescription 312 before completing each purchase order 630.

To validate the medical cannabis prescription 312, the processor 112 can be operated to determine whether the medical cannabis prescription 312 has expired. The processor 112 can check the prescription expiry date 342 provided in the medical cannabis prescription 312 or if the prescription expiry date 342 is not available, determine the prescription expiry date 342 from the prescription date 330 based on the relevant regulations. When the prescription expiry date 342 has not passed, the processor 112 can be operated to complete the second purchase order 630b.

In some embodiments, to complete the second purchase order 630b, the processor 112 can be operated to cancel the registration of the medical cannabis prescription 312 with the first licensed seller 424a, and then to automatically register the medical cannabis prescription 312 with the second licensed seller 424d. When cancelling the registration of the medical cannabis prescription 312 with the first licensed seller 424a, the processor 112 can be operated to determine the cancellation requirements associated with the first licensed seller 424a and to comply with the cancellation requirements. The processor 112 can determine the registration requirements associated with the second licensed seller 424b, and comply with the registration requirements for the second licensed seller 424b.

It will be appreciated that numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description and the drawings are not to be considered as limiting the scope of the embodiments described herein in any way, but rather as merely describing the implementation of the various embodiments described herein.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that the term “coupled” used herein indicates that two elements can be directly coupled to one another or coupled to one another through one or more intermediate elements.

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example and without limitation, the programmable computers (referred to herein as computing devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some embodiments, the communication interface may be a network communication interface. In embodiments in which elements are combined, the communication interface may be a software communication interface, such as those for inter-process communication (IPC). In still other embodiments, there may be a combination of communication interfaces implemented as hardware, software, and combination thereof.

Program code may be applied to input data to perform the functions described herein and to generate output information. The output information is applied to one or more output devices, in known fashion.

Each program may be implemented in a high level procedural or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloadings, magnetic and electronic storage media, digital and analog signals, and the like. The computer useable instructions may also be in various forms, including compiled and non-compiled code.

Various embodiments have been described herein by way of example only. Various modification and variations may be made to these example embodiments without departing from the spirit and scope of the invention, which is limited only by the appended claims. Also, in the various user interfaces illustrated in the drawings, it will be understood that the illustrated user interface text and controls are provided as examples only and are not meant to be limiting. Other suitable user interface elements may be possible.

1. An electronic medical cannabis prescription fulfillment system comprising:
an interface component to receive a medical cannabis prescription issued for a patient by a healthcare practitioner;
a storage component to store user data associated with each user account of one or more user accounts of the prescription fulfillment system and product data associated with each medical cannabis product available via the prescription fulfillment system; and
a processor operable to:
identify a user account registered with the prescription fulfillment system corresponding to the patient;
generate an alert to one of the patient and the healthcare practitioner when no user account corresponding to the patient is identified, otherwise, automatically verify patient data received via the medical cannabis prescription with the user data stored in association with the user account in the storage component;
display at a user interface at least one portion of the product data of each medical cannabis product available for purchase;
receive a first purchase order directly from the patient to purchase a first medical cannabis product from a first licensed seller;
complete the first purchaser order;
subsequent to completing the first purchase order, receive a second purchase order directly from the patient to purchase a second medical cannabis product from a second licensed seller different from the first licensed seller; and
in response to receiving the second purchase order, automatically validate the medical cannabis prescription, and complete the second purchase order when the medical cannabis prescription is validated.

2. The system of claim 1, wherein the processor is operable to:
in response to receiving the second purchase order, determine whether the medical cannabis prescription is expired to validate the medical cannabis prescription.

3. The system of claim 1, wherein the processor is operable to:
trigger the patient to create the user account with the prescription fulfillment system when no user account corresponding to the patient is identified.

4. The system of claim 1, wherein the processor is operable to:
trigger the healthcare practitioner to notify the patient to create the user account with the prescription fulfillment system when no user account corresponding to the patient is identified.

5. The system of claim 3, wherein the processor is operable to:
receive a new account request from the patient to create the user account; and
in response to receiving the new account request, display at the user interface one or more account creation user interfaces for receiving at least some of the user data.

6. The system of claim 5, wherein the processor is operable to:
receive a portion of the user data from at least one of the healthcare practitioner and the medical cannabis prescription.

7. (canceled)

8. The system of claim 1, wherein the processor is operable to:
compare the patient data with the stored user data to identify one or more discrepancies; and
automatically notify the healthcare practitioner of the one or more discrepancies when identified.

9. (canceled)

10. The system of claim 1, wherein the processor is operable to:
automatically order the selection of medical cannabis products for the patient based on at least one of the user data, prescription data received from the medical cannabis prescription, and product data associated with each medical cannabis product.

11. The system of claim 10, wherein the processor is operable to automatically order the selection of medical cannabis products for the patient based on a medical condition identified in the medical cannabis prescription.

12. The system of claim 10, wherein the processor is operable to:
determine a historical medical cannabis usage from the user data stored in a storage component accessible via a network; and
automatically order the selection of medical cannabis products for the patient based on the historical medical cannabis usage.

13. (canceled)

14. (canceled)

15. (canceled)

16. A computer-implemented method for operating an electronic medical cannabis prescription fulfillment system, the method comprising:
receiving a medical cannabis prescription issued for a patient by a healthcare practitioner; and
operating a processor to:
identify a user account registered with the prescription fulfillment system corresponding to the patient;
generate an alert to one of the patient and the healthcare practitioner when no user account corresponding to the patient is identified, otherwise, automatically verify patient data from the medical cannabis prescription with the user data stored in association with the user account in a storage component;
display at a user interface at least one portion of the product data of each medical cannabis product available for purchase;
receive a first purchase order directly from the patient to purchase a first medical cannabis product from a first licensed seller;
complete the first purchaser order;
subsequent to completing the first purchase order;
receive a second purchase order directly from the patient to purchase a second medical cannabis product from a second licensed seller different from the first licensed seller; and
in response to receiving the second purchase order, automatically validate the medical cannabis prescription and complete the second purchase order when the medical cannabis prescription is validated.

17. The method of claim 16, wherein operating the processor to automatically validate the medical cannabis prescription comprises:
determining whether the medical cannabis prescription is expired.

18. The method of claim 16, wherein operating the processor to generate the alert to one of the patient and the healthcare practitioner when no user account corresponding to the patient is identified comprises:
triggering the patient to create the user account with the prescription fulfillment system.

19. The method of claim 16, wherein operating the processor to generate the alert to one of the patient and the healthcare practitioner when no user account corresponding to the patient is identified comprises:
triggering the healthcare practitioner to notify the patient to create the user account with the prescription fulfillment system.

20. The method of claim 18, wherein operating the processor to generate the alert to one of the patient and the healthcare practitioner when no user account corresponding to the patient is identified comprises:
receiving a new account request from the patient to create the user account; and
in response to receiving the new account request, displaying at the user interface one or more account creation user interfaces for receiving at least some of the user data.

21. The method of claim 20 further comprises operating the processor to:
receive a portion of the user data from at least one of the healthcare practitioner and the medical cannabis prescription.

22. (canceled)

23. The method of claim 16, wherein operating the processor to automatically verify the patent data comprises:
comparing the patient data with the stored user data to identify one or more discrepancies; and
automatically notifying the healthcare practitioner of the one or more discrepancies when identified.

24. (canceled)

25. The method of claim 16, wherein operating the processor to display the at least one portion of the product data of each medical cannabis product available for purchase to the patient comprises:
automatically ordering the selection of medical cannabis products for the patient based on at least one of the user data, prescription data received from the medical cannabis prescription, and product data associated with each medical cannabis product.

26. The method of claim 25, wherein operating the processor to automatically order the selection of medical cannabis products for the patient comprises:
automatically ordering the selection of medical cannabis products for the patient based on a medical condition identified in the medical cannabis prescription.

27. The method of claim 25, wherein operating the processor to automatically order the selection of medical cannabis products for the patient comprises:
determining a historical medical cannabis usage from the user data stored in a storage component accessible via a network; and
automatically ordering the selection of medical cannabis products for the patient based on the historical medical cannabis usage.

28. (canceled)

29. (canceled)

30. (canceled)

31. (canceled)
